(19) United States
(12) Patent Application Publication
(10) Pub. No.: US 2025/0177280 A1
(43) Pub. Date: Jun. 5, 2025

(54) COSMETIC SUNSCREEN WITH HIGH WET SKIN UV PROTECTION

(71) Applicant: BEIERSDORF AG, Hamburg (DE)

(72) Inventor: Richard PRESTI, Kinnelon, NJ (US)

(21) Appl. No.: 18/529,036

(22) Filed: Dec. 5, 2023

(51) Int. Cl. A61K 8/81; A61K 8/34; A61K 8/37; A61K 8/40; A61K 8/85; A61Q 17/04

(52) U.S. Cl. CPC A61K 8/8164; A61K 8/34; A61K 8/37; A61K 8/40; A61K 8/85; A61Q 17/04

(57) ABSTRACT

A cosmetic preparation which comprises Acrylates/Octylacrylamide Copolymer and one or more propanediol copolymers exhibits an enhanced wet skin sunscreen factor.

### BACKGROUND OF THE INVENTION

### 1. Field of the Invention

The present invention relates to a cosmetic preparation which comprises a combination of Acrylates/Octylacrylamide Copolymer and one or more propanediol copolymers.

### 2. Discussion of Background Information

The trend away from pale complexion towards “healthy, athletic tanned skin” has continued for many years. To achieve this, people expose their skin to solar radiation since this causes pigment formation in terms of melanin formation. However, the ultraviolet radiation of sunlight also has a harmful effect on skin. In addition to acute damage (sunburn), long-term harm such as an increased risk of skin cancer arises on excessive irradiation with light in the UVB range (wavelength: 280-320 nm). Excessive exposure to UVB and UVA radiation (wavelength: 320-400 nm) additionally leads to a weakening of the elastic and collagen fibers of the connective tissue. This leads to numerous phototoxic and photoallergic reactions and results in premature ageing of the skin.

To protect the skin, a series of photoprotective filter substances which may be used in cosmetic preparations have therefore been developed. In the majority of industrialized countries, these UVA and UVB filters are compiled in the form of positive lists to the cosmetics regulation.

The plethora of commercially available sunscreens should not however obscure the fact that these preparations of the prior art have a series of disadvantages.

Most sunscreens are employed in the vicinity of water or during a sporting activity (perspiration), for which reason the water resistance of such formulations is of particular importance. A water-resistant sunscreen protects the user not only after bathing, but also protects her against sunburn during bathing. It is therefore advisable for those who participate in water sports and, for example, swim, surf or snorkel, and in particular children, who often play for hours at or in the water, to also protect the skin against overly intense and excessive solar irradiation with a sun product which adheres well and can only be rinsed off with difficulty by water and perspiration.

Apart from water resistance, the application of a sunscreen to the wet skin is an important factor for a successful UV protection. For this reason, in 2016, a new factor of resistance appeared in claims and was demanded by consumers: wet skin application. This refers to the capability of sun care products to be applied directly to wet skin, e.g., immediately after emerging from water—with a guarantee of unreduced sun protection. In other words, it enables the user to skip the step of drying the skin before applying sunscreen. This factor and its determination was described e.g., by Elise Delamour, Sébastien Miksa and Dominique Lutz, HelioScreen Labs, Creil, France in “Wet Skin Factor for Sunscreens: An in vitro Method, Part I,” Cosmetics & Toiletries, July 6^{th} 2016. The disclosure of this document is incorporated by reference herein.

Many of the popular sunscreen products are ethanol-based sprays. Because of their ethanol base, these products have an only low water resistance. To improve the water resistance of these sprays, most of the products on the market contain octinoxate and oxybenzone as highly waterproof UV filters. Especially for high SPF values it seemed to be necessary to use these UV filters. On the other hand, for different reasons there is a strong need to replace these UV filters. A public discussion about coral bleach, environmental hazards etc., whether justified or not, leads to a consumer desire for sunscreen sprays that are substantially free of octinoxate and oxybenzone.

In view of the foregoing, it would be advantageous to have available an ethanol-based sunscreen spray which is free of octinoxate and oxybenzone.

Another way of improving the water resistance of cosmetic formulas is based on the addition of film forming polymers. However, there is a limit to how much film forming polymer can be added to a formulation due to solubility limits, aesthetics or performance. More specifically, the performance of a film former can decrease at higher concentrations if the film former becomes brittle and “cracks”.

In view of the foregoing, it would also be advantageous to have available an ethanol-based sunscreen spray with a high SPF and a high water-resistance as well as a satisfactory performance and attractive sensoric properties.

### SUMMARY OF THE INVENTION

Surprisingly it has been found that a cosmetic preparation comprising a combination of Acrylates/Octylacrylamide Copolymer and one or more propanediol copolymers overcomes the disadvantages of the prior art.

Although US 2015/0157543 A1 is known to those skilled in the art, this document, which is incorporated by reference herein, is unable to suggest the approach of the present invention.

The cosmetic preparation of the invention preferably comprises the Acrylates/Octylacrylamide Copolymer in a concentration from about 0.01% to about 10.0% by weight, based on the total weight of the preparation. Even more preferred is a concentration of from about 2% to about 5% by weight, based on the total weight of the preparation. Acrylates/Octylacrylamide Copolymer is available under the tradename “Dermacryl 79” from the Nouryon.

The cosmetic preparation of the invention preferably comprises one or more propanediol copolymers in a (total) concentration of from about 0.01% to about 10.0% by weight, based on the total weight of the preparation. Even more preferred is a concentration of from about 2% to about 5% by weight, based on the total weight of the preparation.

In one embodiment, the cosmetic preparation comprises a propanediol copolymer which is based on a dimer dilinoleate/propanediol copolymer. For example, the propanediol copolymer may comprise one or both of bis-octyldodecyl dimer dilinoleate/propanediol copolymer and bis-dodecylhexadecyl dimer dilinoleate/propanediol copolymer. These copolymers are available under the Tradenames “Cosmosurf DDG-20” and “Cosmosurf DDG-28” from Surfatech.

A cosmetic preparation which comprises bis-octyldodecyl dimer dilinoleate/propanediol copolymer is preferred.

In a further embodiment of the preparation of the invention the ratio by weight of Acrylates/Octylacrylamide Copolymer and the one or more propanediol copolymers is from 3:1 to 2:1.

In a further embodiment, the preparation is a sunscreen preparation and/or a spray formulation (preferably both).

In a further embodiment, the cosmetic preparation comprises one or more (and preferably all or at least three or two) of avobenzone, octisalate, homosalate, and octocrylene.

A preferred concentration of avobenzone in the preparation is from about 2% to about 4% by weight, based on the total weight of the preparation.

A preferred concentration of octisalate in the preparation is from about 3% to about 7% by weight, based on the total weight of the preparation.

A preferred concentration of homosalate in the preparation is from about 12% to about 18% by weight, based on the total weight of the preparation.

A preferred concentration of octocrylene in the preparation is from about 7% to about 14% by weight, based on the total weight of the preparation.

In one embodiment, the preparation contains ethanol, preferably in a concentration of from about 40% to about 60% by weight, based on the total weight of the preparation.

In a further embodiment, the preparation according to the invention comprises ethylhexyl methoxycrylene (2-propenoic acid, 2-cyano-3-(4-methoxyphenyl)-3-phenyl-, 2-ethylhexylester), preferably in a concentration of from about 1.8% to about 2.5% by weight, based on the total weight of the preparation. This substance is available from Hallstar under the tradename “SolaStay S1”.

In a further embodiment, the preparation of the invention comprises Polyester-8 (hexanedioic acid, polymer with 2,2-dimethyl-1,3-propanediol, 3-[(2-cyano-1-oxo-3,3-diphenyl-2-propenyl)oxy]-2,2-dimethylpropyl 2-octyldodecyl ester), preferably in a concentration of from about 2.5% to about 3.5% by weight, based on the total weight of the preparation. This substance is available from Hallstar under the tradename “Polycrylene”.

In a further embodiment, the preparation of the invention comprises butyloctyl salicylate (benzoic acid, 2-hydroxy-, 2-butyloctyl ester), preferably in a concentration of from about 4% to about 6% by weight, based on the total weight of the preparation. This substance is available from Hallstar under the tradename “Hallbrite BHB”.

In a further embodiment, the preparation of the invention comprises diethylhexyl syringylidenemalonate (and) caprylic/capric triglyceride (propanedioic acid, 2-[(4-hydroxy-3,5-dimethoxyphenyl)methylene]-, 1,3-bis(2-ethylhexyl) ester), preferably in a concentration of from about 0.8% to about 1.3% by weight, based on the total weight of the preparation. This substance is available from EMD Performance Materials/Rona under the tradename “Oxynex ST”.

The present invention further provides a method for enhancing the wet skin sunscreen factor of a cosmetic sunscreen composition. The method comprises adding Acrylates/Octylacrylamide Copolymer and one or more propanediol copolymers to the composition in an amount which enhances the wet skin sunscreen factor of the composition.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those of skill in the art how the several forms of the present invention may be embodied in practice.

### Comparative Experiment

The effect in accordance with the invention can be substantiated with the following experiment by way of example:

The Water resistance was measured in accordance with the procedure set forth in 21 CFR 201.327 as follows:

### Sunscreen Application

(i) Test site. Test sites are locations on each subject's back, between the beltline and the shoulder blades (scapulae) and lateral to the midline, where skin responses to UV radiation are determined. Responses on unprotected skin (no test material applied) and protected skin (sunscreen test product(s) or SPF standard applied) are determined at separate unprotected and protected test sites, respectively. Test sites should be randomly located in a blinded manner. Each test site should be a minimum of 30 square centimeters and outlined with indelible ink.
(ii) Test subsite. Test subsites are the locations to which UV radiation is administered within a test site. At least five test subsites should receive UV doses within each test site. Test subsites should be at least 0.5 square centimeters (cm²) in area and should be separated from each other by at least 0.8 cm. Each test subsite should be outlined with indelible ink.
(iii) Applying test materials. Apply the sunscreen test product and the SPF standard at 2 milligrams per square centimeter (mg/cm²) to their respective test sites. Use a finger cot compatible with the sunscreen to spread the product as evenly as possible.
(iv) Waiting period. Wait at least 15 minutes after applying a sunscreen product before exposing the test sites to UV radiation as described in paragraph (i) (5)) of this section. For water resistant sunscreen products, proceed with the water resistance testing procedure described in paragraph (i) (7) of this section after waiting at least 15 minutes.

### UV Exposure

(i) Definition of minimal erythema dose (MED). The minimal erythema dose (MED) is the smallest UV dose that produces perceptible redness of the skin (erythema) with clearly defined borders at 16 to 24 hours after UV exposure. The MED for unprotected skin (MEDu) is determined on a test site that does not have sunscreen applied. The MED for protected skin (MEDp) is determined on a test site that has sunscreen applied. An MEDp is determined for the SPF standard (ssMEDp). An MEDp is determined for the sunscreen test product (tpMEDp).
(ii) UV exposure for initial MEDu. For each test subject, administer a series of UV radiation doses expressed as J/m²-eff (as determined according to paragraph (a)(2) of this section) to the test subsites within an unprotected test site using an accurately calibrated solar simulator. Select doses that are a geometric series represented by 1.25n (i.e., each dose is 25 percent greater than the previous dose).
(iii) UV exposure for final MEDu, ssMEDp, and tpMEDp. For each subject, determine the final MEDu, ssMEDp, and tpMEDp by administering a series of five UV doses to the appropriate test sites. The middle dose (X) in each of these dose series (i.e., the third dose) should equal the initial MEDu times the expected SPF. Note that the expected SPF equals 1 and 16.3 for the final MEDu and ssMEDp, respectively. The remaining UV doses in the series depend upon the expected SPF value of the sunscreen test product(s).

For products with an expected SPF less than 8, administer UV doses that increase by 25 percent with each successive dose (i.e., 0.64×, 0.80×, 1.00×, 1.25×, and 1.56×). For products with an expected SPF from 8 to 15, administer UV doses that increase by 20 percent with each successive dose (i.e., 0.69×, 0.83×, 1.00×, 1.20×, and 1.44×). For products with an expected SPF higher than 15, administer UV doses that increase by 15 percent with each successive dose (i.e., 0.76×, 0.87×, 1.00×, 1.15×, and 1.32×).
(iv) Evaluation of test subsites. In order that the person who evaluates the test subsites is not biased, he/she should not be the same person who applied the sunscreen drug product to the test site or administered the UV doses. After UV doses are administered, all immediate responses should be recorded. These may include an immediate darkening or tanning, typically grayish or purplish in color, which fades in 30 to 60 minutes; an immediate reddening at the subsite, due to heating of the skin, which fades rapidly; and an immediate generalized heat response, spreading beyond the subsite, which fades in 30 to 60 minutes. After the immediate responses are noted, each subject should shield the exposed area from further UV radiation until the MED is determined. Determine the MED 16 to 24 hours after

UV exposure. Because erythema is evaluated 16 to 24 hours after UV exposure, the final MEDu, ssMEDp, and tpMEDp are typically determined the day following determination of the initial MEDu. Evaluate the erythema responses of each test subsite using either tungsten or warm white fluorescent lighting that provides at least 450 lux of illumination at the test site. For the evaluation, the test subject should be in the same position as when the test site was irradiated.

### Determination of SPF

(i) Calculate an SPF value for each test subject (SPFi) as follows:
   SPFi=MEDpMEDu
(ii) Calculate the mean
   SPF(SPF⁻)
and the standard deviation(s) from the SPFi values. Calculate the standard error (SE), which equals s/Vn (where n equals the number of subjects who provided valid test results). Obtain the t value from Student's t distribution table corresponding to the upper 5-percent point with n−1 degrees of freedom. Determine the labeled SPF value, which equals the largest whole number less than
   SPF⁻−(t*SE),

In order for the SPF determination of a test product to be considered valid, the SPF value of the SPF standard should fall within the standard deviation range of the expected SPF (i.e., 16.3±3.43).

### Determination of Water Resistance

The following procedure should be performed in an indoor fresh water pool, whirlpool, and/or hot tub maintained at 23 to 32° C. Fresh water is clean drinking water that meets the standards in 40 CFR part 141. The pool and air temperature and the relative humidity should be recorded.
(i) Water resistance (40 minutes). The labeled SPF should be determined after 40 minutes of water immersion using the following procedure:
(A) Step 1: Apply the sunscreen as described in paragraph (d) of this section.
(B) Step 2: Perform moderate activity in water for 20 minutes.
(C) Step 3: Rest out of water for 15 minutes. Do not towel test site(s).
(D) Step 4: Perform moderate activity in water for 20 minutes.
(E) Step 5: Allow test sites to dry completely without toweling.
(F) Step 6: Apply the SPF standard as described in paragraph (d) of this section.

Step 1. Expose test sites to UV doses as described in paragraph (e) of this section.
(ii) Water resistance (80 minutes). The labeled SPF should be determined after 80 minutes of water immersion using the following procedure:
(A) Step 1: Apply the sunscreen as described in paragraph (d) of this section.
(B) Step 2: Perform moderate activity in water for 20 minutes.
(C) Step 3: Rest out of water for 15 minutes. Do not towel test site(s).
(D) Step 4: Perform moderate activity in water for 20 minutes.
(E) Step 5: Rest out of water for 15 minutes. Do not towel test site(s).
(F) Step 6: Perform moderate activity in water for 20 minutes.
(G) Step 7: Rest out of water for 15 minutes. Do not towel test site(s).
(H) Step 8: Perform moderate activity in water for 20 minutes.
(I) Step 9: Allow test sites to dry completely without toweling.
(J) Step 10: Apply the SPF standard as described in paragraph (d) of this section.
(K) Step 11: Expose test sites to UV doses as described in paragraph (e) of this section.

The following compositions A to C were tested according to the procedures set forth above.

### Composition A (Comparative)

Part A Ingredients

| | |
|---|---|
| Ethylhexyl Methoxycrylene | 2.30 |
| Tocopherol | 0.25 |
| Ethylhexyl Salicylate | 5.00 |
| Butyl Methoxydibenzoylmethane | 3.00 |
| Homosalate | 15.00 |
| Octocrylene | 10.00 |
| Polyester-8 | 3.00 |
| Butyloctyl Salicylate | 5.00 |
| Diethylhexyl Syringylidenemalonate | 1.12 |
| Ascorbyl Palmitate | 0.01 |

Part B Ingredients

| | |
|---|---|
| Ethanol | 51.55 |
| Fragrance | 0.10 |
| Diisostearoyl Polyglyceryl-3 Dimer | 1.67 |
| Dilinoleate and C12-15 Alkyl Benzoate | |
| Acrylates/Octylacrylamide Copolymer | 2.00 |
| | 100.00 |

### Results:

| Test | Subjects | Mean in vivo SPF value |
|---|---|---|
| 80 min water resistance | 2 | 32.25 |

### Composition B

Part A Ingredients

| | |
|---|---|
| Ethylhexyl Methoxycrylene | 2.30 |
| Tocopherol | 0.25 |
| Ethylhexyl Salicylate | 5.00 |
| Butyl Methoxydibenzoylmethane | 3.00 |
| Homosalate | 15.00 |
| Octocrylene | 10.00 |
| Polyester-8 | 3.00 |
| Butyloctyl Salicylate | 5.00 |
| Diethylhexyl Syringylidenemalonate | 1.12 |
| Ascorbyl Palmitate | 0.01 |

Part B Ingredients

| | |
|---|---|
| Ethanol | 48.22 |
| Fragrance | 0.10 |
| Bis-Octyldodecyl Dimer Dilinoleate/Propanediol Copolymer | 2.25 |
| Acrylates/Octylacrylamide Copolymer | 4.75 |
| | 100.00 |

### Results:

| Test | Subjects | Mean in vivo SPF value |
|---|---|---|
| 80 min water resistance | 10 | 70.5 |

### Composition C

Part A Ingredients

| | |
|---|---|
| Ethylhexyl Methoxycrylene | 2.30 |
| Tocopherol | 0.25 |
| Ethylhexyl Salicylate | 5.00 |
| Butyl Methoxydibenzoylmethane | 3.00 |
| Homosalate | 15.00 |
| Octocrylene | 10.00 |
| Polyester-8 | 3.00 |
| Butyloctyl Salicylate | 5.00 |
| Diethylhexyl Syringylidenemalonate | 1.12 |
| Ascorbyl Palmitate | 0.01 |

Part B Ingredients

| | |
|---|---|
| Ethanol | 48.22 |
| Fragrance | 0.10 |
| Bis-Octyldodecyl Dimer Dilinoleate/Propanediol Copolymer | 2.00 |
| Acrylates/Octylacrylamide Copolymer | 5.00 |
| | 100.00 |

### Results:

| Test | Subjects | Mean in vivo SPF value |
|---|---|---|
| 80 min water resistance | 10 | 60.1 |

What is claimed is:

1. A cosmetic preparation, wherein the preparation comprises (a) Acrylates/Octylacrylamide Copolymer and (b) one or more propanediol copolymers.

2. The cosmetic preparation of claim 1, wherein the preparation comprises (a) in a concentration of from about 0.01% to about 10.0% by weight, based on a total weight of the preparation.

3. The cosmetic preparation of claim 1, wherein the preparation comprises (b) in a concentration of from about 0.01% to about 10.0% by weight, based on a total weight of the preparation.

4. The cosmetic preparation of claim 1, wherein (b) comprises a copolymer based on a dimer dilinoleate/propanediol copolymer.

5. The cosmetic preparation of claim 1, wherein (b) comprises one or both of bis-octyldodecyl dimer dilinoleate/propanediol copolymer and bis-dodecylhexadecyl dimer dilinoleate/propanediol copolymer.

6. The cosmetic preparation of claim 1, wherein (b) comprises bis-octyldodecyl dimer dilinoleate/propanediol copolymer.

7. The cosmetic preparation of claim 1, wherein a ratio by weight of (a):(b) is from 3:1 to 2:1.

8. The cosmetic preparation of claim 1, wherein the preparation is a sunscreen preparation.

9. The cosmetic preparation of claim 1, wherein the preparation is a spray formulation.

10. The cosmetic preparation of claim 1, wherein the preparation further comprises one or more of avobenzone, octisalate, homosalate, octocrylene.

11. The cosmetic preparation of claim 1, wherein the preparation further comprises ethanol.

12. The cosmetic preparation of claim 11, wherein the preparation comprises ethanol in a concentration of from about 40% to about 60% by weight, based on a total weight of the preparation.

13. The cosmetic preparation of claim 1, wherein the preparation further comprises ethylhexyl methoxycrylene.

14. The cosmetic preparation of claim 13, wherein the preparation comprises ethylhexyl methoxycrylene in a concentration of from about 1.8% to about 2.5% by weight, based on a total weight of the preparation.

15. The cosmetic preparation of claim 1, wherein the preparation further comprises Polyester-8.

16. The cosmetic preparation of claim 15, wherein the preparation comprises Polyester-8. in a concentration of from about 2.5% to about 3.5% by weight.

17. The cosmetic preparation of claim 1, wherein the preparation further comprises butyloctyl salicylate.

18. The cosmetic preparation of claim 17, wherein the preparation comprises butyloctyl salicylate in a concentration of from about 4% to about 6% by weight.

19. The cosmetic preparation of claim 1, wherein the preparation is free of octinoxate and oxybenzone.

20. A method of enhancing the wet skin sunscreen factor of a cosmetic sunscreen composition, wherein the method comprises adding to the composition Acrylates/Octylacrylamide Copolymer and one or more propanediol copolymers in an amount which is sufficient to enhance the wet skin sunscreen factor of the composition.
